(19) United States
(12) Patent Application Publication
(10) Pub. No.: US 2006/0173214 A1
(43) Pub. Date: Aug. 3, 2006

(54) Process for the preparation of enantiomerically pure (r) or (s)-5-(2-aminopropyl)-2-methoxybenzenesulfonamide

(76) Inventors: Sushil K Dubey, Ahmedabad, Guajarat (IN); Dharmeshkumar A Patel, Ahmedabad, Gujarat (IN); Dhimant J Patel, Ahmedabad (IN); Mahesh L Rupapara, Ahmedabad, Gujarat (IN); Virendra Kumar Agarwal, Ahmedabad, Gujarat (IN); Kanwal Pandita, Ahmedabad, Gujarat (IN); Pankaj Patel, Ahmedabad, Gujarat (IN)

Correspondence Address: NIXON & VANDERHYE, PC, 901 NORTH GLEBE ROAD, 11TH FLOOR, ARLINGTON, VA, 22203, US

(21) Appl. No.: 10/540,556

(22) PCT Filed: Dec. 26, 2002
(86) PCT No.: PCT/IN02/00244

§ 371 Date: Dec. 13, 2005

(51) Int. Cl. C07C 303/02

(52) U.S. Cl. 564/86

(57) ABSTRACT

The invention relates to a process for the manufacture of optically pure (R) or (S)-5-(2-aminopropyl)-2-methoxybenzenesulfonamide with D-i.e. (2S, 3S) or L-i.e. (2R, 3R)-tartaric acid to form a mixture of diastereomeric salts, separating the diastereomeric salts by fractional crystallization in a mixture of solvent systems and at the specified temperature range and contacting the individual salts so separated with a base to provide said R-(−)-5-(2-aminopropyl)-2-methoxybenzenesulfonamide or S-(+)-5-(2-amino propyl)-2-methoxybenzenesulfonamide.

### FIELD OF THE INVENTION

The present invention relates to a process for the preparation of enantiomerically pure (R) or (S)-5-(2-aminopropyl)-2-methoxybenzenesulfonamide by resolution of (R, S)-5-(2-aminopropyl)-2-methoxybenzenesulfonamide with D-i.e. (2S, 3S) or L-i.e. (2R, 3R)-tartaric acid respectively. The optically pure (R)-(−)-5-(2-aminopropyl)-2-methoxybenzenesulfonamide of formula I is a key intermediate for the -preparation of Tamsulosin of formula II (EP-34432, U.S. Pat. No. 4,703,063) useful in the treatment of patients with symptomatic benign prostatic hyperplasia (BPH).

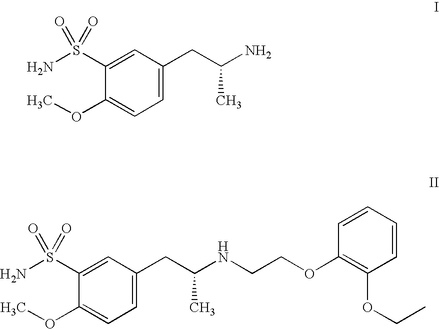

### BACKGROUND OF THE INVENTION

European Patent 257787 describes a method for the preparation of optically pure (R)-(−)-5-(2-aminopropyl)-2-methoxybenzenesulfonamide based on the principle of diastereomeric separation. Thus 5-acetonyl-2-methoxybenzene-sulfonamide (III) as in scheme-1 is reacted with an optically active benzylic amine (IV) under reducing conditions to obtain a diastereomeric mixture (V) (RR: SR) in varying ratio depending upon the reducing agent employed. The material thus obtained is subjected to the following operations:
(i) suspension in a solvent, (ii) heating for a specified time, (iii) cooling, (iv) filtration. This cycle of operations is repeated (at least four times) till an optical purity of 98.0% of VI is achieved.

Compound VI of 98% optical purity is again mixed with water & another solvent such as acetone or acetonitrile. The mixture is heated to reflux, cooled & filtered. The crystals obtained are subjected to repeated cycles of mixing with the solvent, refluxing, cooling & filtration (at least-three times) until desired optical purity of >99.5% is obtained for Compound VII (Scheme-1).

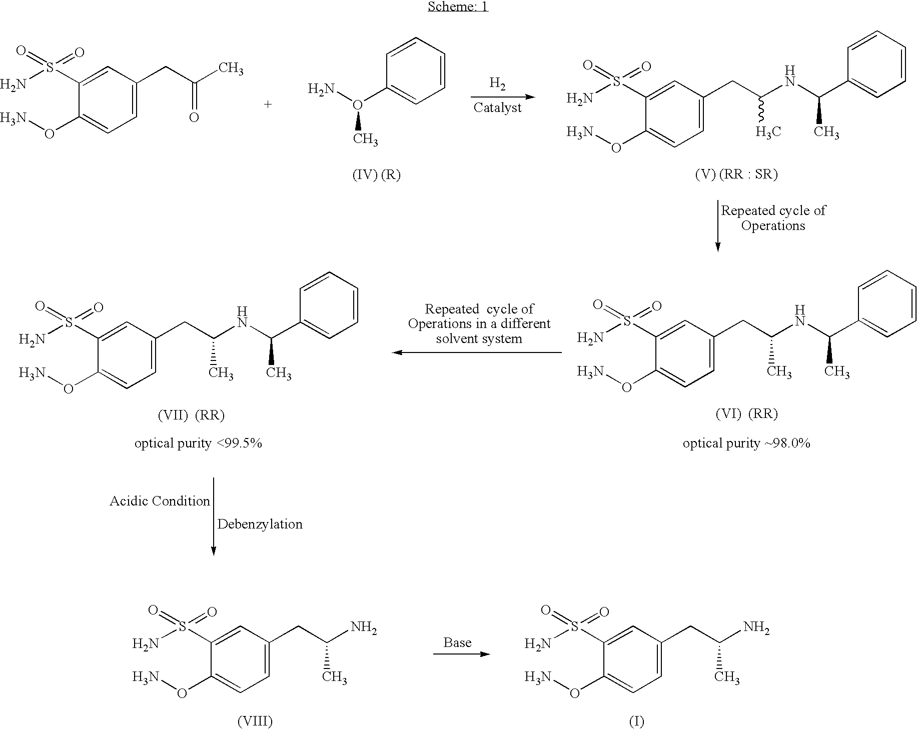

Optically pure diastereomer VII is debenzylated under acidic conditions to obtain R-(−)-5-(2-aminopropyl)-2-methoxybenzenesulfonamide as hydrochloride salt (VIII), which is isolated and treated with a base to obtain desired compound I.

U.S. Pat. No. 4,703,063 relates to novel sulfamoyl-substituted phenethylamine derivatives and the acid addition salts thereof, and more particularly, to novel sulfamoyl-substituted phenethylamine derivatives and the acid addition salts thereof which exhibit a strong .alpha.-adrenergic blocking, action and are useful as an antihypertensive agent and a treating agent for congestive heart failure.

### OBJECTS OF THE INVENTION

It is evident from above that though prior art looks conceptually very good but practically, it is very cumbersome & time consuming. One will have to carry out number of times repeated operations as well as analysis to obtain optically pure I. Therefore, the above mentioned process is not a suitable process to operate for large-scale production.

Accordingly, it is an object of the present invention to provide a plant friendly yet inexpensive process for the manufacture of R-(−)-5-(2-aminopropyl)-2-methoxybenzenesulfonamide (I). This object of the present invention can only be achieved, if one can devise a suitable diastereomeric salt of I whose differential solubility properties could be exploited in a suitable solvent system at an appropriate temperature range to obtain desired optically pure (R)-(−)-5-(2-aminopropyl)-2-methoxybenzenesulfonamide (I) in the minimum possible operations.

### DETAILED DESCRIPTION

Accordingly, the present invention provides a process for the manufacture of optically pure (R) or (S)-5-(2-aminopropyl)-2-methoxybenzenesulfonamide (I), which comprises resolving (R,S)-5-(2-aminopropyl)-2-methoxybenzene-sulfonamide with D- or L-tartaric acid to form a mixture of diastereomeric salts, separating the diastereomeric salts in two stage process in a mixture of solvent systems of the kind such as described at a specified temperature range and contacting the individual salts so separated with base of the kind such as herein described to certain pH range such as herein described to provide said (R)-(−)-5-(2-aminopropyl)-2-methoxybenzenesulfonamide (I) in optically pure form (Scheme-2).

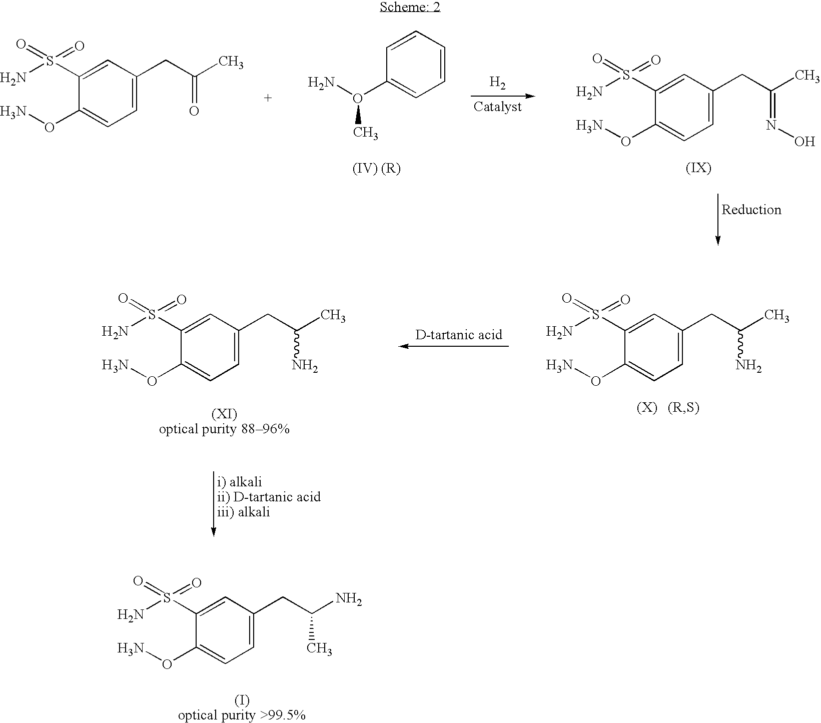

(R, S)-5-(2-aminopropyl)-2-methoxybenzenesulfonamide can be prepared by any known procedure but is preferably prepared by a two step procedure (Scheme-2) starting from 5-acetonyl-2-methoxybenzenesulfonamide (III) converting its keto functional group into oxime (IX) & reducing it with hydrogen under catalytic conditions to obtain the racemic amino compound (X).

Optical purity of more than 99.5% is achieved through two stage procedure. In the first stage racemic 5-(2-aminopropyl)-2-methoxybenzenesulfonamide (X) is reacted with D-tartaric acid to form the salt (XI) having a melting point between 189-195° C. Salt obtained is cleaved with alkali and pH is adjusted between 9.5-10. Substantially resolved amine is obtained as a crystalline solid. This is again treated with D-tartaric acid followed by desalting to obtain optically pure R (−)-5-(2-aminopropyl)-2-methoxybenzenesulfonamide (I).

Preferably, the ratio of (R, S)-5-(2-aminopropyl)-2-methoxybenzenesulfonamide to the D (−) tartaric acid is 1:1 to 1:1.1 mole. The diastereomeric salt formation as well as resolution is preferably carried out in the same solvent system, but these operations can also be performed in two different solvents.

It has been observed that the resolution of 5-(2-aminopropyl)-2-methoxybenzenesulfonamide tartarate is largely governed by the polarity of the solvent system used. The solvent system preferred is a combination of alcoholic solvents such as methanol, ethanol, propanol having 5-20% (v/v) of polar solvents such as amidic solvents e.g. dimethylformamide, N-methyl-2-pyrrolidone; dimethylsulfoxide or water. Though water alone can also be used for salt formation and resolution at room temperature, however in order to obtain optimum yield and optical purity, it's used in combination with alcoholic solvents.

Temperature plays a major role in obtaining the optically pure R-(−)-5-(2-aminopropyl)-2-methoxybenzenesulfonamide. It has been observed that if the resolution is carried out at 50-70° C., the desired product is obtained in a higher optical purity. Temperature between 60-65° C. provides the best results and is therefore preferable.

The reaction time may vary between 4 to 26 hrs after the addition of the amine to the tartaric acid; however under optimal reaction conditions, reaction time of 6 to 8 hrs is preferred to obtain the optimum optical purity as well as yield.

The separated solid salt (R amine: D acid) from the reaction mass is isolated by filtration. Optically purified diastereomeric salt is treated with a base preferably sodium hydroxide to bring pH 9.5-10 to obtain free R-(−)-5-(2-aminopropyl)-2-methoxybenzenesulfonamide.

From the mother liquor obtained from second stage filtration, 5-(2-aminopropyl)-2-methoxybenzenesulfonamide is recovered in ˜90% optical purity and is used in next cycle of resolution to enhance the productivity. It can also be resolved separately if so desired.

The resolving agent D-tartaric acid can be recovered from the aqueous part by usual methods known in the literature and can be recycled for resolution.

Besides D-tartaric acid, L-tartaric acid can also be used as a resolving agent. The resolution of (R, S)-5-(2-aminopropyl)-2-methoxybenzenesulfonamide using L-tartaric acid is carried out in the same fashion. However in this case, (2S, 3S,S)-diastereomeric salt separates out from the reaction mixture which after desalting gives the S-(+)-5-(2-aminopropyl)-2-methoxybenzenesulfonamide.

The present invention is further described in greater detail as illustrated in non-limiting examples. It should be understood that variations & modifications of the process are possible within the ambit of the invention broadly disclosed herein.

### EXAMPLE: 1

### 5-(2-Hydroxyiminopropyl)-2-methoxybenzenesulfonamide

A mixture of 55 gm 5-acetonyl-2-methoxybenzenesulfonamide, 23.6 gm hydroxylamine hydrochloride, 35.4 gm triethylamine and 275 ml of methanol was stirred at room temperature for 5 hours. Then methanol was distilled out and after distillation 275 ml water was added to the reaction mass. The reaction mixture was stirred for 1 hour at room temperature and separated solid was filtered. 49.5 gm 5-(2-hydroxyiminopropyl)-2-methoxy benzenesulfonamide was obtained.

Melting point: 193-4° C.; ¹H NMR: 1.7 δ (—CH₃), 3.5 δ (—CH₂—), 3.95 δ (—O—CH₃), 7.1, 7.4, 7.7 δ (aromatic-H); m/e: 259.1 (M+H)⁺.

### EXAMPLE: 2

### 5-(2-aminopropyl)-2-methoxybenzenesulfonamide

A mixture of 30 gm 5-(2-hydroxyiminopropyl)-2-methoxybenzene sulfonamide, 10.5 gm Raney nickel (wet) and 450 ml methanol was shaken in a Parr apparatus at room temperature under 4 kg hydrogen pressure for 8 hours. At the end of reaction, Raney nickel was filtered off. Filtrate was distilled out to obtain 27.6 gm 5-(2-aminopropyl)-2-methoxybenzenesulfonamide.

Melting point: 180° C.; ¹H NMK: 0.9 δ (—CH₃), 2.9 δ (—CH—), 2:5 δ (—CH₂—), 3.8 δ (—O—CH₃), 7.1, 7.3, 7.5 6 (aromatic-H); m/e: 245.4 (M+H)⁺.

### EXAMPLE: 3

### R-(−)-5-(2-aminopropyl)-2-methoxybenzenesulfonamide tartarate

50 gm 5-(2-aminopropyl)-2-methoxybenzenesulfonamide was dissolved in a solvent mixture of 500 ml of methanol and water (9.5: 0.5 v/v) on heating. 33.8 gm D (−) tartaric acid was added slowly at 65° C. in the reaction mixture, maintained the temperature for 6 hours. The crystals were collected by filtration, washed with methanol and dried to provide 30.40 gm tartarate salt of R-(−)-5-(2-aminopropyl)-2-methoxybenzenesulfonamide tartarate.

Meltin [α]^{D}²⁵ (c=1.0, H₂O): −19.4°

### EXAMPLE: 4

### R-(−)-5-(2-aminopropyl)-2-methoxybenzenesulfonamide tartarate

25 gm of 5-(2-aminopropyl)-2-methoxybenzenesulfonamide was dissolved in 250 ml of a solvent mixture of methanol and dimethyl sulfoxide (8.0: 2.0 v/v) on heating. 16.9 gm D (−) tartaric acid was added 60° C., cooled it to 50° C. and maintained it for 26 hours. The crystals were collected by filtration, washed with methanol and dried to provide 7.6 gm tartarate salt of R-(−)-5-(2-aminopropyl)-2-methoxy benzene sulfonamide.

Melting point: 196-7° C. (d). [α]_{D}²⁵ (c=1.0, H₂O): −20.6°

### EXAMPLE: 5

### R-(−)-5-(2-aminopropyl)-2-methoxybenzenesulfonamide tartarate

250 gm of R-(−)-5-(2-aminopropyl)-2-methoxybenzenesulfonamide was dissolved in 2125 ml of methanol and 425 ml dimethyl formamide on heating. 169 gm D (−) tartaric acid was added slowly at 60° C., maintained temperature for 6 hours. The crystals were filtered off. The crystals, which were separated out (wet), were taken in 750 ml of methanol and stirred for half an hour. The solid was filtered off and washed with methanol, thereby affording 193.3 gm. tartarate salt of R-(−)-5-(2-aminopropyl)-2-methoxybenzenesulfonamide.

Melting point: 193-4° C.(d); [α]_{D}²⁵ (c=1.0, H₂O): −19.16°

### EXAMPLE: 6

### R-(−)-5-(2-aminopropyl)-2-methoxybenzenesulfonamide tartarate

25 gm of 5-(2-aminopropyl)-2-methoxybenzenesulfonamide was dissolved in 250 ml of solvent mixture of isopropyl alcohol and water (8:2 v/v) on heating 16.9 gm D (−) tartaric acid was added at 70° C., maintained it for 6 hours. The crystals were collected by filtration, washed with isopropyl alcohol and dried to provide 17.9 gm tartarate salt of R-(−)-5-(2-aminopropyl)-2-methoxy-benzenesulfonamide.

Melting point: 189-90° C.(d); [α]_{D}²⁵ (c=1.0, H₂O): −17.4°

### EXAMPLE: 7

### R-(−)-5(2-aminopropyl)-2-methoxybenzenesulfonamide tartarate

25 gm of 5-(2-aminopropyl)-2-methoxybenzenesulfonamide was dissolved in 250 ml solvent mixture of methanol and dimethyl sulfoxide (9.5:0.5 v/v) on heating. 16.9 gm D (−) tartaric acid was added at 60° C., maintained it for 6 hours. The crystals were collected by filtration, washed with methanol and dried to provide 18.9 gm tartarate salt of R-(−)-5-(2-aminopropyl)-2-methoxy- benzenesulfonamide.

Melting point: 188-9° C.(d);

### EXAMPLE: 8

### R-(−)-5-(2-aminopropyl)-2-methoxybenzenesulfonamide tartarate

25 gm 5-(2-aminopropyl)-2-methoxybenzenesulfonamide was dissolved in 250 ml solvent mixture of methanol and dimethyl sulfoxide (9:1 v/v) on heating. 16.9 gm D (−) tartaric acid was added at 60° C., maintained it for 6 hours. The crystals were collected by filtration, washed with methanol and dried to provide 15.1 gm tartarate salt of R-(−)-5-(2-aminopropyl)-2-methoxybenzenesulfonamide.

Melting point: 193-4° C.(d); [α]_{2D}²⁵ (c=1.0, H₂O): −18.70°

### EXAMPLE: 9

### R-(−)-5-(2-aminopropyl)-2-methoxybenzenesulfonamide tartarate

25 gm 5-(2-aminopropyl)-2-methoxybenzenesulfonamide was dissolved in 250 ml solvent mixture of methanol and N-methyl 2-pyrrolidone (9.0:1.0 v/v) on heating. 16.9 gm D (−) tartaric acid was added at 60° C., maintained it for 6 hours. The crystals were collected by filtration, washed with methanol and dried to provide 15.0 gm tartarate salt of R-(−)-5-(2-aminopropyl)-2-methoxy-benzenesulfonamide.

Melting point: 192-3° C. (d); [α]_{D}²⁵ (c=1.0, H₂O): −18.49°

### EXAMPLE: 10

### R-(−)-5-(2-aminopropyl)-2-methoxybenzenesulfonamide tartarate

25 gm 5-(2-aminopropyl)-2-methoxybenzenesulfonamide was dissolved in 250 ml solvent mixture of methanol and N-methyl 2-pyrrolidone (8.0:2.0 v/v) on heating. 16.9 gm D (−) tartaric acid was added at 60° C., maintained it for 6 hours. The crystals were collected by filtration, washed with methanol and dried to provide 13.3 gm tartarate salt of R-(−)-5-(2-aminopropyl)-2-methoxybenzenesulfonamide.

Melting point: 193-4° C.(d); [α]_{D}²⁵ (c=1.0, H₂O): −18.57°

### EXAMPLE: 11

### R-(−)-5-(2-aminopropyl)-2-methoxybenzenesulfonamide tartarate

25 gm 5-(2-aminopropyl)-2-methoxybenzenesulfonamide was dissolved in 375 ml solvent mixture of methanol and water (9.5:0.5 v/v) on heating. 16.9 gm D (−) tartaric acid was added at 60° C., maintained it for 14 hours. The crystals were collected by filtration, washed with methanol and dried to provide 10.4 gm tartarate salt of R-(−)-5-(2-aminopropyl)-2-methoxybenzenesulfonamide

Melting point: 196-7° C. (d); [α]_{D}²⁵ (c=1.0, H₂O): −20.29°

### EXAMPLE: 12

### R-(−)-5-(2-aminopropyl)-2-methoxybenzenesulfonamide tartarate

25 gm 5-(2-aminopropyl)-2-methoxybenzenesulfonamide was dissolved in 375 ml methanol and 37.5 ml N,N-dimethylformamide on heating. 16.9 gm D (−) tartaric acid was added at 60° C., maintained it for 14.5 hours. The crystals were collected by filtration, washed with methanol and dried to provide 11.7 gm tartarate salt of R-(−)-5-(2-aminopropyl)-2-methoxy benzenesulfonamide.

Melting point: 197-8° C.(d); [α]_{D}²⁵ (c=1.0,H₂O): −20.7°

### EXAMPLE: 13

### R-(−)-5-(2-aminopropyl)-2-methoxybenzenesulfonamide tartarate

25 gm 5-(2-aminopropyl)-2-methoxy benzenesulfonamide was dissolved in 250 ml methanol and 12.5 ml N,N-dimethylformamide on heating. 16.9 gm D(−) tartaric acid was added at 60° C., maintained the temperature for 6 hours. The crystals were collected by filtration, washed with methanol and dried to provide 16.1 gm tartarate salt of R-(−)-5-(2-aminopropyl)-2-methoxybenzenesulfonamide.

Melting point: 191-2° C. (d); [α]_{D}²⁵ (c=1.0 H₂O): −19.11°

### EXAMPLE: 14

### R-(−)-5-(2-aminopropyl)-2-methoxybenzenesulfonamide tartarate

25 gm 5-(2-aminopropyl)-2-methoxybenzenesulfonamide was dissolved in 175 ml solvent mixture of methanol and water (9:1 v/v) on heating. 16.9 gm D(−) tartaric acid was added at 60° C., maintained the temperature at 65° C. for 14 hours. The crystals were collected by filtration, washed with methanol and dried to provide 15.4 gm tartarate salt of R-(−)-5-(2-aminopropyl)-2-methoxy-benzenesulfonamide.

Melting point: 194-5° C.(d); [α]_{D}²⁵ (c=1.0, H₂O): −19.12°

### EXAMPLE: 15

### R-(−)-5-(2-aminopropyl)-2-methoxybenzenesulfonamide

To 75 gm tartarate salt (as obtained in example: 5: m.p.:193-4° C.) of R-(−)-5-(2-aminopropyl)-2-methoxybenzene sulfonamide in 75 ml water was added 40% sodium hydroxide solution, so as to adjust pH between 9.5-10. Reaction mixture was stirred for 1 hour at room temperature. 38.7 gm of R-(−)-5-(2-aminopropyl)-2-methoxybenzenesulfonamide was collected after filtration.

Melting point: 170-1° C.; [α]_{D}²⁵ (c=1.0, H₂O): −14.49°

### EXAMPLE: 16

### R-(−)-5-(2-aminopropyl)-2-methoxybenzenesulfonamide tartarate

25 gm of R-(−)-5-(2-aminopropyl)-2-methoxybenzenesulfonamide (m.p.:170° C.) was dissolved in solvent mixture of 250 ml methanol and 50 ml N,N-dimethylformamide on heating. 16.9 gm D (−) tartaric acid was added at 60° C., maintained the temperature for 6 hours. The crystals formed were collected by filtration, washed with methanol. The crystals, which were collected out (wet), were stirred for ½ an hour with 75 ml methanol. The solid was filtered off and washed with methanol, affording 32.7 gm tartarate salt of R-(−)-5-(2-aminopropyl)-2-methoxybenzenesulfonamide.

Melting point: 198° C.(d); [α]_{D}²⁵ (c=1.0, H₂O): −20.5°

### EXAMPLE: 17

### R-(−)-5-(2-aminopropyl)-2-methoxybenzenesulfonamide tartarate

10 gm of R-(−)-5-(2-aminopropyl)-2-methoxybenzenesulfonamide (m.p.=170° C. ) was dissolved in 100 ml solvent mixture of methanol and water (9.3:0.7 v/v) on heating, 6.8 gm D(−) tartaric acid was added at 65° C., maintained the temperature for 6 hours. The crystals formed were collected by filtration, washed with methanol and dried to provide 8.9 gm tartarate salt of R-(−)-5-(2-aminopropyl)-2-methoxybenzenesulfonamide.

Melting point: 198° C.(d); [α]_{D}²⁵ (c=1.0, H₂O): −20.6°

### EXAMPLE: 18

### R-(−)-5-(2-aminopropyl)-2-methoxybenzenesulfonamide tartarate

10 gm of R-(−)-5-(2-aminopropyl)-2-methoxybenzenesulfonamide (m.p.: 172° C.)was dissolved in 100 ml a solvent mixture of methanol and water (9:1 v/v) on heating, 6.8 gm D(−) tartaric acid was added at 65° C., maintain it for 8 hours. Crystals formed were collected by filtration washed with methanol and dried to provide 10.9 gm tartarate salt of R-(−)-5-(2-aminopropyl)-2-methoxy benzenesulfonamide.

Melting point: 198° C.(d); [α]_{D}²⁵ (c=1.0, H₂O): −20.6°

### EXAMPLE: 19

### R-(−)-5-(2-aminopropyl)-2-methoxybenzenesulfonamide

To 10 gm tartarate salt (as obtained in example 16 ) of R-(−)-5-(2-aminopropyl)-2-methoxybenzene sulfonamide in 10 ml water was added 40% sodium hydroxide solution, so as to adjust pH between 9.5-10. Reaction mixture was stirred for 1 hour at room temperature. 6.0 gm of R-(−)-5-(2-aminopropyl)-2-methoxybenzenesulfonamide was collected after filtration. Melting point: 16: [α]_{D}²⁵ c=1.07, Methanol): −17.11° (lit: −17.3°)

1. A process for manufacture of optically pure (R) or (S)-5-(2-aminopropyl)-2-methoxybenzenesulfonamide by using a suitable diasteromeric salts of (R, S)-5-(2-aminopropyl)-2-methoxybenzenesulfonamide whose differential solubility properties exploited in a suitable solvent system at a suitable temperature range obtains desired optically phase (R)-(−)-5-(2-aminopropyl)-2 methoxtbenzensSulfonamide, said process comprising resolving (R, S)-5-(2-aminopropyl)-2-methoxybenzenesulfonamide with D-or L-tartaric acid to form a mixture of diastereomeric salts, separating the diastereomeric salts in any known manner in the presence of inert organic solvents of the kind such as herein described and contacting the individual salts so separated with base of the kind such as herein before described to provide said R-(−)-5-(2-aminopropyl)-2-methoxybenzenesulfonamide or S-(+)-5-(2-aminopropyl)-2-methoxybenzenesulfonamide, wherein the ratio of the polar solvent to alcoholic solvent varies from 5 to 20% (v/v), and said said resolution is carried out in a temperature range of 50-70° C.

2. A process as claimed in claim 1 wherein the ratio of (R, S)-5-(2-aminopropyl)-2-methoxybenzenesulfonamide to tartaric acid is in the range of 1:1 to 1:1.1.

3. A process as claimed in claim 1 wherein resolution is carried out in two stages in presence of a solvent system consisting of alcoholic solvents coupled with varying ratios of polar solvents such as acidic solvents like dimethylformamide, N-methyl-2-pyrrolidone or dimethylsulfoxide or water.

4. A process as claimed in claim 1 wherein said resolution is preferably carried out at a temperature in the range of 60-65° C.

5. A process as claimed in claim 1 wherein the said reaction time is between 4 to 26 hrs.

6. A process as claimed in claim 1 wherein the inert organic solvent used for separating the diastereomeric salts to individual salts is selected from the group consisting of one or more of methanol, ethyl alcohol, propyl alcohol, water, dimethylformamide, N-methyl-2-pyrrolidone, dimethylsulfoxide.

7. A process as claimed in claim 1 wherein the base is sodium hydroxide & the pH for isolation of free base is 9.5-10.

8. A process as claimed in claim 1, whereby melting point of tartarate salt of more than 188° C. is obtained after first stage operations.

9. A process as claimed in claim 1, whereby an optically purity of more than >99.5% is obtained after second stage operations.

10. A process as claimed claim 1, wherein 5-(2-aminopropyl)-2-methoxybenzenesulfonamide is obtained in more than 90% optical purity from the second stage mother liquor.

11. A process whereby racemic (R, S) 5-(2-aminopropyl)-2-methoxybenzenesulfonamide is prepared in two step synthesis as shown in scheme: 2 from 5-acetonyl-2-methoxybenzenesulfonamide.
